(19) United States
(12) Patent Application Publication
(10) Pub. No.: US 2015/0093786 A1
(43) Pub. Date: Apr. 2, 2015

(54) Laboratory apparatus and method of using a laboratory apparatus

(71) Applicant: Eppendorf AG, Hamburg (DE)

(72) Inventors: Andreas THIEME, Hamburg (DE); Judith LUCKE, Hamburg (DE); Rusbeh GOECKE, Hamburg (DE); Manfred EBERS, Hamburg (DE); Helmut KNOFE, Hamburg (DE); Harald ANDRULAT, Hamburg (DE)

(21) Appl. No.: 14/040,419

(22) Filed: Sep. 27, 2013

(51) Int. Cl. G01N 35/00; B01L 7/00; C12Q 1/68

(52) U.S. Cl. CPC G01N 35/00584; C12Q 1/686; B01L 7/52; G01N 2035/00277
USPC 435/91.2; 422/510

(57) ABSTRACT

The invention is related to a laboratory apparatus and method for the automated processing of liquid samples, in particular for the program controlled handling of liquid samples, having an electronic control device, which is adapted to process a program code for the program controlled processing of fluid samples, at least one processing space for receiving the fluid samples to be processed, at least one electronically controllable sample processing device for performing at least one program controlled process step on at least one sample, which is arranged in the processing space, at least one electronically controllable decontamination device for cleaning at least a part of the processing space, wherein the decontamination device is configured to be controlled by the control device and the control device is configured to digitally control the decontamination device.

The invention relates to a laboratory apparatus for the automated processing of liquid samples, in particular for the program controlled handling of liquid samples. The invention further relates to method for using a laboratory apparatus for the automated processing of fluid samples.

Said laboratory apparatus are used in chemical, biological, biochemical, medical and forensic laboratories for processing fluid laboratory samples, in particular liquid samples, with high efficiency. Processing steps are automatized by said kind of laboratory apparatus, which otherwise would have been performed manually. This way, the speed, precision and reliability of sample treatment can be enhanced.

The purpose of a sample treatment, typically, is to measure, analyze, process and/or modify a sample, or, in particular, to systematically process a plurality of samples, e.g. by running a predefined method of treatment. The treatment can include changing a physical parameter like the sample's volume, temperature or homogeneity. The treatment can further include changing a chemical or biochemical property of the sample(s), for example to modify the composition of the sample, e.g. the dilution or purification of DNA or RNA, or the set up and performance of a PCR (polymerase chain reaction). A sample treatment can require to separate, divide or to dilute the sample(s), and in particular, to dose a sample volume, to transport a sample, and to distribute a sample, e.g. using pipetting techniques. Contents of a sample can be analyzed by said apparatus, or new samples can be provided by, for example, providing a chemical and/or enzymatic reaction in the sample. In particular with regard to the processing of DNA or RNA, or their building blocks, said laboratory apparatus are useful and commonly used to acquire a lot of information within an acceptable time of analysis by performing a plurality of processing steps in an automated way.

A laboratory apparatus has a processing space, which is adapted to receive at least one sample vessel, containing a sample, or to receive a plurality of samples, and to receive other accessory components for processing the samples. Usually, the processing space is manually loaded with the sample vessels containing the samples to be automatically processed. The processing space includes a processing area, which can be arranged to provide several processing stations.

The positions of the processing stations are programmed to the programmed control device of the apparatus such that the positions can be addressed, for transporting articles, in particular samples and/or sample vessels, between the different processing stations. For example, liquid samples can be transported between the sample vessels at a first processing station to the sample vessels at a second processing station. Said transport can be achieved by a program controlled robotic transport device, which has a fluid transfer device, e.g. a pipetting tool, for aspirating the fluid samples at the first processing station and distributing it at the second processing station.

Transporting the fluid samples in this way introduces a risk of sample leakage, which can lead to the formation of satellite drops and aerosols, and thus to a contamination of the processing space, in particular of the processing area, or to a cross-contamination of other samples, which are positioned in the processing space. Moreover, the processing space is exposed to the environment, while the user manually prepares the processing space by assembling the sample vessels, which also introduces contamination. Therefore, some laboratory apparatus are equipped with a decontamination device, e.g. an air filtering device or a UV-light, which can be manually activated to decontaminate the processing area. The filtering of air by using a ventilator in combination with a filter can be performed, generally, during the operation of automatically processing the samples, while this, usually, is not the case for the use of UV-light, because many samples, in particular biological or biochemical samples, are damaged by the UV-light. Therefore, the decontamination of the processing space by UV-light is, usually, initiated by a user before starting—or after finishing—a method of sample treatment. The decontamination of the processing space improves the reliability and efficiency of sample treatments, and is therefore generally useful, if it is performed in the appropriate way.

Regarding an aspect of the present invention, it was observed that the users of a laboratory tend to avoid activating the decontamination devices, either, because this requires additional time, which seems to decrease the productivity in using the laboratory apparatus, or because the operation of the decontamination device of the conventional apparatus was not possible in the way desired by the user.

It is the object of the present invention to provide an improved laboratory apparatus and an improved method for using a laboratory apparatus.

The object is met by the laboratory apparatus according to claim 1 and the method according to claim 11.

The laboratory apparatus according to the invention is configured for the automated processing of fluid samples, in particular for the program controlled handling of liquid samples. The laboratory apparatus has an electronic control device, which is adapted to process a program code for the program controlled processing of fluid samples, the laboratory apparatus also has a processing space for receiving the fluid samples to be processed, it also has at least one electronically controllable sample processing device for performing at least one program controlled process step on at least one sample, which is arranged in the processing space, and it has at least one electronically controllable decontamination device for cleaning at least a part of the processing space. The decontamination device is configured to be digitally controlled by the control device and the control device is configured to at least temporarily digitally control the decontamination device.

Therefore, the laboratory apparatus, hereinafter also referred to as “apparatus”, according to the invention, allows a much more flexible approach regarding the decontamination of the processing space. The decontamination process is digitally controlled by the apparatus, using the control device, and therefore the decontamination of the processing space can be performed in a more controlled way, thereby relieving the user of the duty to repeatedly manually trigger the decontamination during the work time.

This way, the productivity and the quality of sample processing is enhanced. Moreover, preferred embodiments of the apparatus according to the invention are directed to constructional aspects for improving the decontamination of the processing space of the apparatus.

The laboratory apparatus has at least one processing space, which is adapted to receive at least one sample vessel, containing a sample, or to receive a plurality of samples, and to receive other accessory components for processing the samples. Usually, the processing space is manually loaded with the sample vessels containing the samples to be automatically processed. The processing space includes a processing area, which can be arranged to provide multiple processing stations. Preferably, the processing area is substantially planar. This simplifies the decontamination of the processing area. It is possible that a substantially planar processing area has means for aligning lab-ware components. Such means can be a pin or recess, located, preferably, at a processing station. A lab-ware component is a device, which is configured to be used with the laboratory apparatus as an optional, modular device. A lab-ware component can be, for example, a sample holder device, which can be temperature controlled or not, a storage container, a storage container for pipetting or dispensing tips, a holding device for at least one tool device. Some of the lab-ware components are described below, in detail.

Preferably, a processing space has a number N_{PS} of processing stations, wherein N_{PS}, is chosen from the numbers between 1 and 21, wherein the embodiments with 4, 6, 9, 12 and 15 processing stations are preferred. However, it is also possible and preferred that N_{PS}>20.

Preferably, the area of a processing station is substantially planar and has a rectangular outer contour, with two opposing sides a and two opposing sides b, wherein a and b have, respectively, dimensions of of about I_low<=a, b<=I_high, wherein preferably I_low and I_high are chosen, respectively from {5; 8; 10; 12; 15; 20} cm. Preferably, the area of a processing station is adapted to have at least the outer dimensions of a standard microtiter plate, e.g. 127,76 mm×85,48 mm×14,35 mm, as defined in the industrial norms ANSI Standards (ANSI/SBS 1-2004, ANSI/SBS 2-2004, ANSI/SBS 3-2004, ANSI/SBS 4-2004). The areas of different processing stations, preferably, have the same size and shape. This simplifies the automated use of the processing stations, in particular the automated decontamination.

Preferably, the laboratory apparatus is configured to have two or three processing spaces, which can be separated, respectively, in particular by a separation device, which spatially introduces a barrier between at least two vicinal processing spaces. Different processing spaces are, preferably, adapted to perform, at least in part, different process steps. This can optimize the sample treatment. For example, a first processing space can be adapted for performing the steps of purification of PCR samples, which are required to perform a PCR. In a second processing space, a PCR mastermix can be produced, and in a third processing space, the PCR can be run. Optionally, in a fourth processing space, the PCR samples can be analyzed by a method for characterizing a PCR sample, e.g. by detecting the concentration of DNA or RNA or their parts by fluorescence markers. Providing more than on processing space allows to adapt the decontamination effort, in particular to adapt the intensity, schedule and/or selection of the desired decontamination device(s), in dependence on the requirements of the processing steps, which are performed in the different processing spaces.

The laboratory apparatus has an electronic control device, also referred to as “control device”, which is adapted to process a program code for the program controlled processing of fluid samples. At the same time, the control device is configured to also digitally control the at least one decontamination device. This means that the control device has digital processing device, e.g. a processor or CPU or a microprocessor, for controlling digital signals, which control the operation of the at least one decontamination device, in particular the schedule of operation and/or non-operation, the absolute time, durance, intensity of operation, and/or which control the operation in dependence on other parameters, in particular in dependence on at least one operational parameter, which indicates, for example, a status of the configuration of the apparatus.

Preferably, the control device controls the automated processing of samples using a control program. This way, the treatment of the sample is program controlled. Preferably, the control device also controls the operation of the decontamination device using a control program, which is preferably the same control program which also controls the automated processing of samples. The control program controls the operation of at least one device of the apparatus, in particular the operation of the sample processing device and the decontamination device. Preferably, the control device, in particular the control program, uses at least one control parameter to control the operation of at least one device of the apparatus. Moreover, the control device, in particular the control program, is preferably configured to use at least one program parameter for defining at least one control parameter. The implementation of the program control, in particular the software implemented functionality of the apparatus, is described in the following.

The program controlled treatment of the sample means that the process of treatment is substantially controlled automatically, in particular according to the specifications of a computer program, in particular the control program. Any user input is not required for the automatic treatment of the sample, at least after having received the basic user defined program parameters, which are required to run the automatic treatment.

The digital control of the decontamination device means that the signals, which control the decontamination device, are controlled by a control program, in particular by a decontamination program, which, respectively, is run by the control device. Said signals can be analogous and/or digital. For example, digital signals can be output by a CPU of the control device and can be converted to analogous signals by a D/A converter, which outputs analogous signals, which start and/or stop and/or adjust a decontamination device, which can be controlled by analogous signals.

A program parameter is understood to be a variable, according to which a computer program or subprogram can receive input values, which define the data flow of the program. The settings of the program parameters influence, in particular, the result of the program. A program parameter can be a parameter required to be input by the user, which is then called a user defined parameter or user defined program parameter. Such a parameter, usually, is required for the automatic treatment of samples, e.g. the automated processing of the samples according to a treatment method. Further program parameters, which are not required to be input by the user, can be derived from the user defined parameters or can be determined in another way.

Preferably, the at least one program parameter, in particular the user defined parameter, is related to at least one physical and/or characteristic quantity of the following group, which are relevant for the treatment of at least one sample by a sample processing device: number of samples to be processed, dilution factor, target volume, source and/or destination position) of at least one sample in a sample vessel holder or in a microtiter plate or other sample vessel device, sample temperature or rates of modification of the sample temperature, time period, point in time, mixing time, PCR-temperature levels and cycling times, time period for magnetic treatment, in particular magnetic separation, pressure and exposition time in a vacuum chamber of the laborator apparatus, parameters, which activate or deactivate a feature, sub-program or function, and the like.

A program parameter can be a program parameter for controlling the at least one decontamination device, in particular for controlling at least one time period and/or the intensity of activity of at least one decontamination device, for controlling the switching on and/or switching off of the at least one decontamination device according to a predetermined sequence of work steps, which may differ in time and intensity or which may refer to different decontamination devices, e.g. the combined use of a UV-light device and an air cleaning device. The program parameter for controlling the decontamination device can also be related to the method for determining the parameter, e.g. to the way of determining the parameter by the user or automatically.

The control device, in particular the control program, preferably controls the automated processing of samples according to a treatment method by using a program module. A program module is understood to have the conventional meaning. In particular, a program module is a closed functional unit of a software, having a sequence of processing steps and data structures. The content of a program module can be related to a calculation or processing of data, which has to be repeated frequently. A program module can include an encapsulation of data processing by separating the interface for data exchange and the implementation of the data processing. The interface of a program module can define the data elements, which are required to be input to the data module, thereby defining the result of the processing of data by the module. A program module can be called as a function or a subprogram by another program, e.g. the control program. The program module runs a sequence of processing steps, wherein a processing step can be related to the processing of at least one sample, to the control of the decontamination device and/or the call of a decontamination program, for example. The program module can provide as a result output of the output data which are provided to the higher program. A program module can call other program modules, thereby forming a hierarchical structure of a control program. The data structure, which is defined in a program module, can be provided for automatically creating new program modules, or to create a method program, which is explained in the following.

A control program is understood to be an executable computer program, which effects the desired automatic treatment of at least one sample in dependence on program parameters, in particular user defined parameters. The control device controls the treatment in dependence on program parameters. The control device, preferably, generates control parameters for controlling the devices of the apparatus, in particular the at least one sample processing device and/or the decontamination device.

A method program is a control program, which is specific for a type of sample treatment and/or which is specific for a defined treatment of at least one sample. A method program controls the automatic or semi-automatic process of a sample treatment according to a type of sample treatment or according to a defined treatment, wherein the treatment is preferably chosen by the user.

Preferably, the apparatus, in particular with its method programs, allows the user to select the type of treatment, which should be used for an automatic, or respectively, semi-automatic treatment of the samples. The apparatus, in particular with its method programs, is further configured to let the user select the program parameters for the type of treatment for defining the treatment. Preferably, the apparatus is further configured to let the user select at least one program parameter for controlling the decontamination device.

Examples of typical types of treatments of the laboratory apparatus, and their names, cited in quotation marks, are described in the following:

Regarding the purification of nucleic acids:
“MagSep Blood gDNA”: Implements the protocol for purification of genomic DNA from whole blood using the Eppendorf MagSep Blood gDNA kit.;
“MagSep Tissue gDNA”: Implements the protocol for purification of genomic DNA from fresh tissue, cell cultures, yeast and bacteria using the Eppendorf MagSep Tissue gDNA kit.

“MagSep Viral DNA/RNA”: Implements the protocol for purification of viral RNA or DNA from cell-free body fluids using the Eppendorf MagSep Viral DNA/RNA kit.;

### Regarding PCR-Applications:

“Compose Mastermix”: Create one or more PCR mastermixes from pre-mixes or single components (buffer, polymerase, dNTPs, primers, probes, etc.). In particular, the software automatically calculates the required program parameters, e.g. the required volume of each component respective for each vessel.
“Normalize Concentrations”: Dilution of DNA/RNA samples to obtain an equal concentration in all samples. In particular, program parameters, e.g. concentrations data, may be entered manually or can be imported from a file;
“Create Dilution Series”: Serial dilution of one or more DNA/RNA standards to create a calibration curve for quantitative PCR;
“Setup Reactions”: Creation of complete reaction setups by combining samples with one or more mastermixes. Optionally, replication of reactions can be created as well.

Other types of treatments can be provided or can be fully or at least in part defined by the user. A type of treatment can be undefined with regard to one or more program parameters, which can be related to sample volume, sample concentration, sample number, and the like. Preferably, the method programs automatically choses at least one program parameter automatically in dependence on the at least one user defined parameter. This way, the user is unburdened from entering values for those program parameters, which can be derived from the at least one user defined parameter. For example, if a user defined target concentration is desired, the apparatus, in particular with its method program(s), can automatically calculate the control parameters, which define the amount of solvent required for diluting a certain volume of a mastermix, which define the tools, consumables and/or mixing steps required for the dilution treatment, and the like.

Preferably, the apparatus automatically selects the set of program parameters, which corresponds to the type of treatment chosen by the user. The set of program parameters can contain the user defined parameters and/or further program parameters. The further program parameters can be automatically determined by the apparatus in dependence on the treatment, and/or in dependence on the user defined parameters. The further program parameters can be stored in a data memory device of the apparatus. The set of program parameters is, preferably, optimized by the apparatus, e.g. regarding processing time and/or the management of consumables, such that the user preferably does not need to have special knowledge on said optimization processes and its programming. Based on the set of program parameters, the control parameters may be automatically derived, which control the at least one sample processing device and the at least one decontamination device.

The set of program parameters can define the accessory components required for a treatment, e.g. the sample vessels, the transport vessels, the processing tools, e.g. a pipetting tool, a magnetic separation tool, a sample mixer tool, or a thermostatic and/or thermal cycler tool, and/or consumables.

Preferably, the set of program parameters contains at least one program parameter for controlling the decontamination device. The at least one program parameter for controlling the decontamination device can be predetermined and/or can be stored in the data memory device of the apparatus. It is possible that the control device is configured, in particular regarding a specific method program, to apply at least one predetermined program parameter for controlling the at least one decontamination device. The at least one program parameter for controlling the decontamination device can be a default parameter, in particular regarding a specific method program, and/or the control device can be configured to ask a user-confirmation of the at least one parameter and/or to allow a modification of the at least one parameter.

A decontamination device can be controlled by activating or deactivating the decontamination device, or by adjusting or amending the intensity of the operation of the decontamination device, e.g., by adjusting or amending the intensity of a UV-light source or the number of revolutions of the ventilator of an air cleaning device. The control device can comprise a closed loop control with at least one control loop for controlling the intensity of the operation of the decontamination device, which enhances the reproducibility of the decontamination effect. The control parameter, which preferably controls the operation of the decontamination device, can be the actuating variable of the closed loop.

Preferably, the control device, in particular the control program, more particular a method program, is configured to control a decontamination device, in particular according to a predetermined decontamination program. Preferably, the control program is configured to control the at least one decontamination device in dependence on at least one program parameter, in particular at least one user defined parameter. This way, the user is unburdened from adjusting the decontamination device. The activity of the at least one decontamination device is rather optimized by program control. For example, in case of a liquid sample, which has a relatively low viscosity and a higher volume, the probability of the formation of aerosols can be relatively high; in this case, the activity of the at least one decontamination device can be increased, and the risk for (cross-) contamination will thus be further reduced. However, in addition or alternatively, the apparatus can also be configured such that the user can define a control parameter, which defines or influences the control of the at least one decontamination. A control parameter, which defines or influences the control of the at least one decontamination, is referred to as decontamination parameter.

The decontamination program can be a predetermined program, in particular a sub-program, and can optionally be modified by the control program, in particular by a program parameter, by a method program and/or the control device. The decontamination program can be stored in a data memory device of the apparatus. The decontamination program can be configured to control at least one step, preferably multiple steps, of operating the decontamination device. A step of operating the decontamination device can include adjusting or amending the intensity of the operation of the decontamination device, in particular during a predetermined time period or at a predetermined time.

Multiple steps of operating the decontamination device can comprise the step of start the operation of the decontamination device, at least one step of adjusting or amending the intensity of the operation of the decontamination device, and the step of stopping the operation of the decontamination device. Preferably, the decontamination program can provide the operation of at least two decontamination devices, preferably different decontamination devices, for optimizing the overall decontamination effect. The two different decontamination devices are, preferably, a UV-light source and an air cleaning device. Said decontamination devices are complementing one another, because the air cleaning device, in particular, cleans the processing space by a convective transport of contaminating particles out of the processing space, while the UV-light source is capable of decontaminating those areas of the processing space, where the contaminating particles are fixated to the processing space.

The control program controls the operation of the at least one decontamination device, preferably in dependence on control program, in particular a method program, and preferably in dependence on at least one program parameter, preferably a user defined program parameter, preferably in dependence on a time parameter, and/or preferably in dependence on a sensor information of a sensor device of the apparatus.

Preferably, the control device, in particular the control program, uses a method program for defining the control of the decontamination device, in particular a method program, which is configured to define the control of the sample processing device according to a method, which can be selected by the user. The start of the decontamination program, preferably, is dependent on the value of a program parameter. The program parameter can be set automatically, by the apparatus, in particular by control program, or can be user defined. The start of the decontamination program, preferably, is initiated by a control parameter.

Preferably, the control device is configured to automatically run a decontamination program before, substantially directly before, a method program is started. “Directly before” means that the decontamination program is finished and between the end of the decontamination program and the beginning of the method program, no other work steps are performed by the sample processing device. Using the decontamination program before the method program, a sterile processing space is prepared before the actual sample treatment according to the method starts. In case that no decontamination program is being run during the method, the method can be run without being disturbed or interrupted by a further decontamination program.

Preferably, the control device is configured to automatically run a decontamination program after, substantially directly after, a method program was finished. “Directly after” means that the method program is finished, and between the end of the method program and the beginning of the decontamination program, no other work steps are performed by the sample processing device. Using the decontamination program after the method program, a sterile processing space is prepared after the sample treatment according to the method has ended, leaving the processing space sterile for the subsequent sample processing. In case that no decontamination program is run during the method, the method can be run without being disturbed or interrupted by a further decontamination program.

Preferably, the control device is configured to automatically run a decontamination program during a method program is executed. Thereby, a sterile processing space is prepared in between the steps of a sample treatment according to the method. This offers flexibility and numerous configurations of a method program, which advantageously implements at least one decontamination program in the method.

Preferably, the control device has a timer device, and, preferably, is adapted for controlling the processing of the samples and/or the controlling the decontamination device in dependence on a time parameter. The time parameter can include information about an absolute time, e.g. time and/or date, or a time period, e.g. a time period to be applied in relation to a reference time or an event, e.g. an event defined by a control program for controlling the automated processing of samples. The time parameter can be user defined or can be defined by the control device.

In the case that the user defines a parameter directly, e.g. by inputting them via a user interface or selecting them from a pre-stored selection of parameters, the control device does not in general subsequently automatically change the parameter's value. The user defined parameter can be stored in a memory device of the apparatus, in particular after being input by the user via a user-interface of the apparatus, or can be pre-stored in a memory device and can be selected by the user. “User defined” includes, preferably, also the option that the user does indirectly define a first parameter, e.g. by defining a second parameter, which is directly correlated with the first parameter. For example, it is possible that the user selects the second parameter, e.g. by pressing a selection button, for example a hardware- or software button of the apparatus, or a number in a graphically displayed selection menu, which number can be correlated to the second parameter, and that the control device automatically assigns the correlated first parameter in dependence on the second parameter. The correlation can be contained in a data table, which can be stored as digital data table in a digital data storage device, also referred to as memory device, of the apparatus, or respectively, the control device. In case that the control device defines a parameter, in general, the value of the parameter is selected, preferably, by means of the program code, which controls the decontamination device and/or the at least one sample processing device. However, the parameter can also be selected by the control device by another program code or by an electrical circuit.

It is possible, for example, that the control device controls the decontamination device at a predefined absolute point in time, e.g. for switching on and/or switching off and/or amending the operation of the decontamination device at a certain time and/or date, for example during the night or the early morning hours, before the laboratory staff starts working with the apparatus. This way, a decontaminated processing space of the apparatus is provided at a specific time. Hereby, it is preferred that the user has activated the respective automatic scheduled decontamination function and/or has defined, or respectively, selected the absolute time, which preferably is stored in a memory device of the apparatus.

It is also possible, for example, that the control device controls the decontamination device in dependence on a time period. The time period can be user-defined or can be automatically defined. Preferably, the operation of the decontamination device is controlled in dependence on the time period and an absolute point in time, or in dependence on an event. The time period can be at a predefined absolute point in time, e.g. for switching on and/or switching off and/or amending the operation of the decontamination device at a certain time and/or date, after the time period or before the time period, and/or in dependence on more than one time periods, which schedule the activity of the at least one decontamination device. This way, a decontaminated processing space of the apparatus is provided before and/or after and/or between a specific time period or several time periods. Hereby, it is preferred that the user has activated the respective automatic scheduled decontamination function and/or has defined, or respectively, selected the absolute time and/or time period(s), which preferably is/are stored in a memory device of the apparatus.

Preferably, the apparatus has at least one sensor device for sensing at least one operational parameter of the apparatus, and to control the decontamination device in dependence on the at least one operational parameter. The operational parameter can represent, e.g., a status of the configuration of the apparatus, e.g. the detection of an open door element, e.g. by using a Reed switch, or the detection of the position of a surface, in particular the height of a surface. The surface can be part of a lab-ware or consumable, or a liquid. The measurement of the surface can detect and/or identify a lab-ware or consumable, or a liquid. The measurement can detect, if a position in the processing area is occupied by a lab-ware or consumable, or a liquid, or if it is unoccupied and free. The measurement of a surface can be performed by an ultrasonic measurement or by a confocal measurement, which is described by EP 1 288 635 A2. Herein, the height is defined to be measured along the direction of gravity.

The operational parameter can be representing the presence of a user being proximate to the apparatus.

The sensor device measures at least one sensor parameter, and the control of the decontamination device is preferably dependent on the value of the sensor parameter. Thereby, more flexibility is gained for using the automatic decontamination feature of the apparatus. A sensor of the at least one sensor device preferably is an optical sensor, including for example at least one source of radiation, e.g. visible light or infrared radiation, e.g. of a laser or and LED, and at least one detector of radiation, e.g. a photo cell or a photomultiplier. The optical sensor can be adapted to perform a confocal measurement, as described before.

A sensor can be an electrical sensor, in particular a sensor based on electromagnetic induction, a magnetic field sensor, e.g. a hall sensor, and/or a sensor comprising a switch, in particular a mechanical switch, or barometer or hygrometer. The sensor can be configured for measuring a property of the environmental air, e.g. the pressure and/or the humidity and/or the presence and/or concentration of aerosols in the air. Aerosols can be measured optically, for example, e.g. by measuring the light scattering in air of a sensor light, e.g. using the known principle of a so called nephelometer.

Preferably, the apparatus has a housing device, which at least partly or substantially completely encases the at least one processing space of the apparatus. The housing, preferably, has at least one transparent portion or is preferably fully transparent. The material of the housing is preferably nontransparent for UV-light. The material, preferably, is PMMA (polymethylmethacrylat; e.g. Plexiglase®). Preferably, the housing device has at least one opening and at least one door element for closing the at least one opening. A door element can be hinged to the housing or can be a separate part of the housing. The opening allows for accessing the processing space, e.g. when the user manually positions the required components at the starting positions at the processing stations of the processing area. Preferably, a door element has at least one opened position and at least one closed position. In a closed position, it is preferred that at least one ventilation channel, e.g. a gap or opening, is provided between the door element and the housing surrounding the door element. This offers the advantage that the air stream field in the at least one processing space can be influenced in a desired manner, in case that a stream of air is generated, e.g. by a ventilation device. The ventilation channels, which connect the processing space and the environment of the apparatus, may be provided for allowing air exchange. This is advantageous, in particular, if the processing space is pressurized, having a pressure over the environmental pressure. It is preferred that the apparatus controls the pressure in the processing space, at least during the processing of samples. The overpressure prevents contaminants from entering the processing space. Preferably, the overpressure is provided by a ventilation device, e.g. the ventilation device of the air cleaning device, which can be a decontamination device of the apparatus. A door element can, however, also close the opening in a gas-tight manner, e.g. for achieving a high degree of sterility within the processing space.

Preferably, the sensor of the at least one sensor device detects the opening status of the at least one door element of the housing. Preferably, the apparatus is configured to automatically control the decontamination device in dependence on the opening status. For example, overpressure can be generated or adjusted in the processing space if an open door element is detected. The activity of a UV-light device, forming a decontamination device of the apparatus, is preferably automatically prevented, for example, in case that an open door element is detected. This prevents the UV-light from escaping, thereby putting the user at risk.

Moreover, the sensor can be configured to detect the contamination and/or the position and/or the intensity of contamination of a surface, e.g. the surface of a processing area of the processing space. For example, an optical measurement can be used, e.g. a photographic method for evaluating the condition of the surface. Contamination, for example transparent liquids with protein-based contamination, can be detected by using a photographic method using fluorescence light and automatic evaluation of the picture, e.g. in particular by an automatic comparison with a comparison picture, which is free from contamination. Spots of contamination can be detected and, in particular, can be treated by a decontamination device using a local treatment, which, in particular, prevents unnecessary decontamination of clean surfaces.

The detected contamination can be used to automatically start a decontamination program which is optimized for the contamination detected. In particular, the time and/or intensity of the air cleaning can be adjusted to the intensity of detected contamination. Moreover, the time, intensity and/or location of irradiation of a surface can be automatically selected in dependence on the detected contamination.

The sensor device can have a proximity sensor. The proximity sensor can be based on electromagnetic induction, e.g. using the known RFID technique, for detecting that a marked object outside the apparatus is proximate to the apparatus, and located within a detection range. The proximity sensor can be based on a motion sensor, which is arranged, in particular at the apparatus, to detect the presence of a user in a detection range, which can be some meters of distance, e.g. 2.0 m, 1.0 m, 0.5 m, 0.25 m. A decontamination program can be started, for example, if a user enters the detection range of the proximity sensor. Preferably, the air cleaning device is started. However, it is also possible that a UV-light device or another decontamination device is started. This offers a comfortable and efficient way of operating an apparatus with a decontamination device.

A decontamination device is a device, which enhances the decontamination of a target area, e.g. the processing space. Decontamination can be, for example, a sterilization process. “Sterilization” is a term generally referring to any process that eliminates or kills all forms of microbial life, including transmissible agents, such as fungi, bacteria, viruses, spore forms, etc., present on a surface or in a space. Decontamination, in particular sterilization, can be achieved by applying the proper combinations of heat, chemicals, in particular gas composition, steam content, irradiation, high pressure, and filtration.

Preferably, the decontamination device includes an air cleaning device, or is an air cleaning device, which has a ventilation device and a filter device. Preferably, the ventilation device is arranged to transport air from the environment of the apparatus through at least one ventilation pathway to the processing space, which is a space inside the apparatus, in particular shielded from the environment by a housing device. Preferably, the filter device is arranged in the ventilation pathway, for filtering the air which enters the processing space. The filter device can comprise at least one particle filter, in particular a High-Efficiency Particulate Air (HEPA) filter. Such filters, in particular, meet the common HEPA and/or EPA standards

The ventilation device, preferably, comprises at least one ventilator. Preferably, the ventilation device has two or three ventilators, which are arranged in parallel, in particular for generating parallel airstreams. This way, the flow field of air in the processing space is more homogeneous, in particular more laminar. Laminar flow fields allow to more efficiently control the pathways of clean air and also contaminated air in the processing space. Preferably, at least two ventilators, preferably at least three, or more, or all ventilators, can be controlled separately. This way, the air stream field within the at least one processing space can be modified, in particular the direction and/or intensity of the air stream can be locally adjusted. This can help to direct an air stream to one or more areas, which require more intense ventilation, and/or to reduce the air stream in other area(s), where less or no ventilation is required.

The ventilator device can have at least one air guiding device, e.g. a wall, fin, curved elements. The air guiding device can have one or more output openings for letting the air stream out from the at least one opening in the direction of the processing space, or more particular, in a direction which is influenced by the air guiding device of the ventilator device. For example, one ventilator in combination with two or more openings can direct the air stream in two or more different spaces of the at least one processing space, and respectively, in two or more directions. This way, a desired air stream field in the at least one processing space can be defined more flexible.

The apparatus can also have at least one air guiding device, e.g. a wall, fin, curved elements, arranged or arrangeable in the at least one processing space or between processing spaces, for directing the air stream field in the at least on processing space in the desired way. The air guiding device can be program controllable, which allows to automatically configure the air stream field in the desired way. The air guiding device can be mounted in the area, which is vicinal to the processing space, e.g. mounted in the bottom area under the processing area. The air guiding device can be arranged movable, e.g. by means of a motor device, which moves the air guiding device, e.g. under control of the control device and/or the control program, in particular the method program and/or the decontamination program. The air guiding device can also separate two processing spaces, e.g. by forming a vertical wall between the two processing spaces.

Preferably, the processing space has a processing area, forming the bottom of the processing space. Moreover, the processing space is preferably encased by the housing device of the apparatus. Preferably, the processing space is substantially cuboid-shaped, because this allows for an efficient design of the apparatus with a small foot print. However, the housing or parts of the housing can be shaped to improve the laminarity of the flow field of air in the apparatus.

Preferably, the housing element has a top side, which is arranged opposite the processing area. Preferably, the housing element has a back side, which is arranged, in particular, opposite the front side of the housing. The top side and, respectively, the back side of the housing can form a wall separating the processing space from other inside spaces of the apparatus, e.g. the apparatus section containing the electronic control device, and/or at least one decontamination device or at least a part of said devices, or tool devices and/or other components of the apparatus. The control device can also be arranged under the processing space, ontop of the processing space, or on a side of the processing space.

Preferably, a processing space can be considered to be virtually divided in a bottom space and a top space, as well as a front space and a back space. The front space is preferably the space, which is oriented to the user, and which is contacted by the front side of the housing device. The back space is preferably the space, which is oriented away from the user, and which is contacted by the back side of the housing device. The bottom space is preferably the space, which is contacted by the processing area (bottom side) of the housing device. The top space is preferably the space, which is contacted by the top side of the housing device. The top space and the bottom space have, preferably, substantially the same volume, which is preferably substantially cuboid shaped. The front space and the back space have, preferably, substantially the same volume, which is preferably substantially cuboid shaped. In a lateral direction, which can be a horizontal direction, the processing space can be divided in a first space and a second space and/or a third space and/or more spaces, which, in particular, connect the front side and the back side of the processing space. The same definitions can be applicable for at least one additional processing space, which may be present

Preferably, the ventilator device is arranged to connect the at least one ventilation pathway of the ventilation device to the top space of the processing space, in order to generate an air stream from upside to downside of the apparatus. This way, the convective transport of aerosols and other contaminating particles follow substantially the direction of gravity, which improves the laminarity of the flow field. Preferably, the at least one ventilator of the ventilation device is oriented to generate an air stream in a direction substantially perpendicular to the processing area, in particular substantially parallel to gravity, and/or in a direction, which is inclined to the direction of gravity not more or equal to 45°, preferably 35°, preferably 20°, preferably 10°, preferably 5°.

It a particularly preferred aspect of the invention, that the ventilation device has at least one ventilator, in particular multiple, i.e. a number of larger than one, ventilators. In case that multiple ventilators are provided, it is preferred that a first ventilator is arranged to produce a first air stream, which runs through a first section of the processing space and that a second ventilator is arranged to produce a second air stream, which runs through a second section of the processing space, wherein the first section and the second section of the processing space are arranged separately, preferably vicinal. This arrangement results in a combined air stream inside the processing space. Preferably, the air cleaning device has multiple ventilators, which are adapted to be controlled individually by the control device. Preferably, the individual control of the multiple ventilators is performed automatically and program controlled, in particular by running a method program. It is preferred that the user is allowed, in particular during running the method program, to choose at least one user parameter, which controls the activity status, i.e. the on/off status, of one ventilator out of the multiple ventilators. The setup of the activity can be related to specific steps during running a method, e.g. by providing a predetermined activity parameter to each step of the method. It is also preferred that the user is allowed, in particular during running the method program, to choose at least one user parameter, which controls the intensity of a ventilator, in particular, the speed of the ventilator, e.g. measured in rounds per minute, in case that the ventilator is set active.

Preferably, the ventilator device is arranged to connect the at least one ventilation pathway of the ventilation device to the volume of the processing space, which is opposite to the wall having a door element, and/or opposite to the volume of the processing space, which is limited by a wall having a door element. Preferably, the ventilator device is arranged to connect the ventilation pathway of the ventilation device to the back space of the processing space, in order to generate an air stream from back to front. This way, any particles and contaminants are hindered from entering the processing space, which may otherwise enter the processing space through an open front door or through ventilation channels in the front side. The convective transport of aerosols and other contaminating particles through openings in the front side is efficiently prevented.

Preferably, the ventilator device is arranged to connect the at least one ventilation pathway of the ventilation device to the top space of the processing space and also to the back space or the volume of the processing space, respectively, which is opposite to the volume of the processing space, which is limited by a wall having a door element, e.g. the front space, such that the air cleaning device is arranged to connect the at least one ventilation pathway to the intersection volume of the top space and the back space.

This way, the two advantages mentioned before are combined, and an efficient flow field of air inside the processing space can be generated during operation of the ventilation device.

Preferably, the processing area of the processing space has at least one ventilation channel, which connects the processing space with the environment. This way, the flow field of air in the processing space, which is caused by the ventilation device, can locally be guided in a desired direction. Preferably, at least one ventilation channel can be arranged in such an area of a processing station, which requires particular effort for decontamination due to a higher level of contamination. This is the case, for example, for the processing station, which receives the trash, which e.g. contains used transport vessels like pipette tips and can contain residual amounts of samples, which can be the source of contamination and cross-contamination of samples in the processing space. Such a station is preferably arranged in the area of the processing space, where the air flowing in the processing space finally leaves the processing space, preferably the front area. Thereby, contaminants generated at the processing station, which receives the trash, are guided out from the processing space. Preferably, the at least one ventilation channel is arranged in the processing area, in particular at the position of the processing station, which receives a trash container.

The apparatus can be configured to automatically provide a predefined humidity within the processing space by controlling the at least one ventilation device in the required manner. An increased intensity of air stream in the processing space will increase the amount of vapour within the processing space, in case that a vapourizable substance, e.g. a solvent or sample, e.g. water or aqueous solution, is present in the processing space or outside the apparatus.

Preferably, the method program is configured to modify or start or stop the activity of the at least one decontamination device, in particular the ventilation device. For example, during the pipetting of samples or during the ejection of pipetting tips from a pipetting head, the activity of a ventilation device can be temporarily modified (reduced and/or stopped), in order to reduce or even prevent the formation of aerosols or intensified in order to increase the removal (guiding out) of aerosols out of the processing space.

Preferably, the decontamination device includes at least one UV-light device or is a UV-light device, which, respectively, contains at least one UV-light source. The maximum of the intensity of the UV-light spectrum of the UV-light source is preferably located between the wavelengths 240 nm and 290 nm, preferably at a wavelength around 250 nm-260 nm, preferably about 254 nm, which is generally considered to be most efficient for decontamination. Preferably, the UV-light source is based on a low pressure mercury vapor lamp.

Preferably, the UV-light device has at least one UV-LED (Ultraviolett light emitting diode). The UV-LED preferably is configured to emit light in the UVC-wavelength region, in particular at a wavelength between 200 nm to 280 nm, preferably about 254 nm. UV-LEDs are commercially available at the filing date of the present patent application. The use of UV-LEDs offers the following advantages: light is generated efficiently, reliability of the light source is high and maintenance costs are low. Moreover, compact arrangements of the UV-light device can be achieved. Light can be easily directed, e.g. by focusing on a target area or a target volume or by generating parallel light for homogeneous illumination.

Preferably, the UV-light device has at least two UV-LEDs. Thereby, more flexibility is gained regarding the light intensity and the direction of light. Preferably, the UV-light device has at least three, four, five, six, seven, eight, nine or at least ten UV-LEDs. Thereby, said flexibility of dosing and directing the light is respectively gained.

Preferably, the UV-LED is configured to be operated to irradiate a target surface or target volume, in a constant illumination mode or, preferably by choice, in a pulsed operation mode. The target area is, preferably, an area of the processing area. The target volume can also be the air of the ventilation pathway of the ventilation device, in order to irradiate the air entering the processing space, before or after optionally passing a filter device.

Pulsed operation of the UV-LED allows for generating UV light with higher intensities of light than in constant illumination mode.

Preferably, the UV-light device has at least one guiding device for guiding the direction of the UV light of the at least one UV-light source of the apparatus. The guiding device can include at least one optical fiber, at least one lens element, e.g. Fresnel-lens or a condenser lens, at least one optical filter element, at least one mirror element, and the like. A guiding device allows for irradiating a selected area, e.g. a selected area of the processing surface. A contamination can be automatically detected, for example, and the area of contamination can be locally illuminated, thereby protecting the non-contaminated areas, which may contain sensible samples. On the other hand, the local illumination with UV light allows for starting chemical processes, which are triggered, amplified, or completed by UV-light.

Preferably, the apparatus has a tool device, e.g. a pipetting tool device, which can in particular be automatically moved by a robot system of the apparatus. The robot system allows to automatically move the tool device in at least one direction, preferably in at least the z-direction of a Cartesian coordinate system, which preferably corresponds to the vertical direction, preferably also in the x and/or the y-direction of said Cartesian coordinate system. The robot system preferably comprises a stage device for holding a motor driven slide element, which carries a connection element for connecting, e.g., a tool device to the movable slide element. The apparatus and/or the robot system, preferably, is/are configured to use different tool devices, which are preferably configured to perform different tasks.

A tool device can be a pipetting tool device, for transferring a liquid sample into at least one or multiple transport vessel by aspirating the same, e.g. a pipetting tip or dispenser tip. The sample(s) is/are transported to a target position and released by evacuating the transport vessel, using gravity, or by dispensing the sample out from the transport vessel. The apparatus, in particular the pipetting tool device, can be configured to automatically move the pipetting tool device to a processing station, which serves as a storage for sterile transport vessels, can be configured to automatically take up the samples from a processing station, which contains the samples to be treated, and can be configured to automatically transport the sample(s) to a processing station, where the samples are processed, e.g. by applying heating and cooling, magnetic field, mixing the samples, distributing the samples to target container vessels, and the like. A tool device can also be a gripping head, for gripping lab-ware and for transporting and/or applying the same in the at least one processing space.

Preferably, the tool device, in particular the pipetting tool device, has at least one UV-source, preferably, for irradiating at least one spot of contamination, and/or preferably, for irradiating at least one sample in a sample vessel, in particular for irradiating a well in a microtiter plate and/or in a cell culture plate. Since the tool device is movable by the robot system, the desired local target areas for the UV-treatment can be easily addressed.

Preferably, at least one cover element is provided, which can be a cover without an opening or a recess and which, in particular, is intransparent for UV-light. The size of the cover element is preferably corresponding to the size of the area of a processing station. For example, a cover element can be adapted to shield (protect) a standard microtiter plate (MTP) against irradiation, or to shield (protect) another lab-ware against radiation. The cover element can, however, have at least one recess or opening, which is transparent for UV-light. In particular the cover element can have at least one opening which is arrangeable over the area to be protected, e.g. a lab-ware (MTP; plate, vessel etc.) at a processing station, thus encasing the lab-ware there and thereby shielding it from the UV-light.

The cover element is preferably used as a mask for the irradiation of unmasked area and for protecting the masked area from being irradiated. Preferably, the cover element can be used to cover an area to be protected from UV-light, before the decontamination process using UV-light is applied to the area, which contains at least a part of the masked area. Preferably, the cover element is configured to mask the openings of sample vessels in a sample vessel device, e.g. to mask the openings of the wells of a microtiter plate, while other portions of the sample vessel device can be unmasked for receiving UV-light during a decontamination process. This way, UV-light can also be applied locally. The cover element can be configured to be transported and/or positioned by the robot system. This allows to integrate the process of masking an area into the process of the automated sample treatment.

UV light can also be automatically applied during a method program for inputting energy into at least one sample. For example, in case that a chemical reaction is triggered, catalyzed and/or stopped by the irradiation by UV light, the decontamination device can also be used for this purpose.

The laboratory apparatus, preferably, is a desktop apparatus, thereby capable of being placed on the workbench of a laboratory. Preferably, the apparatus is compact in design, the apparatus preferably having a footprint of less than 4.0 m², 2.0 m², 1.5 m² or 1.0 m². The apparatus, in particular the processing space, preferably has a volume of less than 4.0 m³, 2.0 m³, 1.5 m³ or 1.0 m³. Such a relatively small volume allows to most efficiently control the decontamination of the processing space.

The liquid sample, preferably is a laboratory sample, in particular a sample, which is processed and/or measured in a biological, biomedical, medical, forensic, biochemical, chemical and/or pharmaceutical laboratory, which can be, in particular a manufacturing laboratory, and/or a research laboratory, and/or a forensic laboratory. The liquid sample, typically, is an aqueous solution, but can also contain or consist of non-aqueous parts, in particular organic and/or inorganic parts, said parts possibly being fluid, in particular liquid, and or solid and/or gaseous phases. The liquid sample can contain biological liquids, in particular solutions containing biological parts, which biological parts can be, for example, living cells, cell fragments, biological molecules, for example DNA and/or fragments of the DNA and/or other nucleic acids and/or proteins. The liquid sample can be a solution containing living cells, i.e. a cell suspension, or can be a solution containing, or consisting of, blood and/or blood serum, or urine or other liquids from human or animal bodies. The liquid sample can also be a solution containing, or consisting of, pharmaceuticals and/or reaction partners for a chemical reaction, in particular for performing a PCR reaction.

The sample processing device is a device, which handles, in particular automatically, at least one sample according to the input, e.g. the control parameters, from the control device. The sample processing device can comprise the tool device and the robot system, which moves the tool device to the predetermined position. The movement of the tool device and the activity of the tool device are controlled by the control device, in particular by the control parameters, in particular in dependence on the program parameters. The sample processing device is configured for performing at least one program controlled process step on at least one sample. The automated liquid handling of samples, which is preferably performed according to a method of sample treatment chosen by the user, in particular according to a method program, is composed of different process steps, which altogether achieve the desired result of automated handling the sample(s) according to the user defined treatment. For example, a process step can be the positioning of the tool device at a first position in the at least one processing space, another process step can be the uptake of a first volume of a liquid sample at the first position, another process step can be the transport of the first sample volume to a second position in the at least one processing space, another process step can be the release of a second volume of the sample at the second position, another process step can be the dilution, shaking, mixing, magnetic separation, heating, cooling, environmental pressure change, and/or irradiation of the sample, and the like.

The process steps of an automated sample treatment are, preferably, performed sequentially. However, it is possible and preferred that at least two process steps of a sample treatment are performed in parallel. This is possible in particular, if a processing station is configured to perform at least two processing steps, for example, heating and mixing of samples, or heating, magnetically treating and pipetting of samples. Such a multifunctional processing station offers the advantage that any additional effort for transporting of samples between multiple processing stations, which would offer only one or only fewer functions, is reduced. Transportation of liquid samples increases the risk of contamination by sample leakage and requires increased activity of the decontamination device(s). In case that a multifunctional processing station is provided, risk of a contamination of the processing space is reduced. Moreover, the net power of the at least one decontamination device is reduced, because the overall process time is reduced and transporting steps can be avoided, which require a higher performance of the at least one decontamination device.

The invention is further directed to a method of operating a laboratory apparatus, in particular the apparatus according to the invention, for the automated processing of fluid samples, in particular for the program controlled pipetting of liquid samples, the apparatus having an electrical control device, which is adapted to process a program code for the program controlled processing of fluid samples, a processing space for receiving the fluid samples to be processed, at least one electrically controllable sample processing device for performing at least one program controlled process step on at least one sample, which is arranged in the processing space, and at least one electrically controllable decontamination device for cleaning at least a part of the processing space, comprising the step of letting the control device automatically control the at least one decontamination device.

Further preferred embodiments of the method according to the invention of operating a laboratory apparatus can be derived from the description of the preferred embodiments of the laboratory apparatus.

Further preferred embodiments of the apparatus according to the invention and the method according to the invention can be derived from the following description of

### PREFERRED EMBODIMENTS OF THE INVENTION

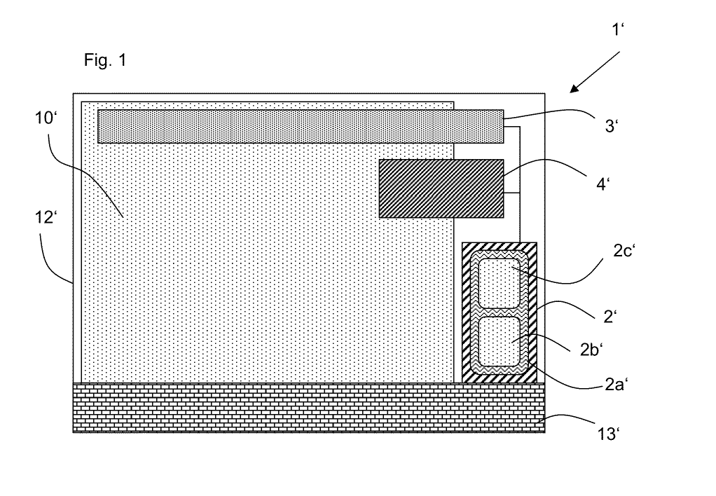

FIG. 1 is a schematic side view of an embodiment of the apparatus according to the invention.

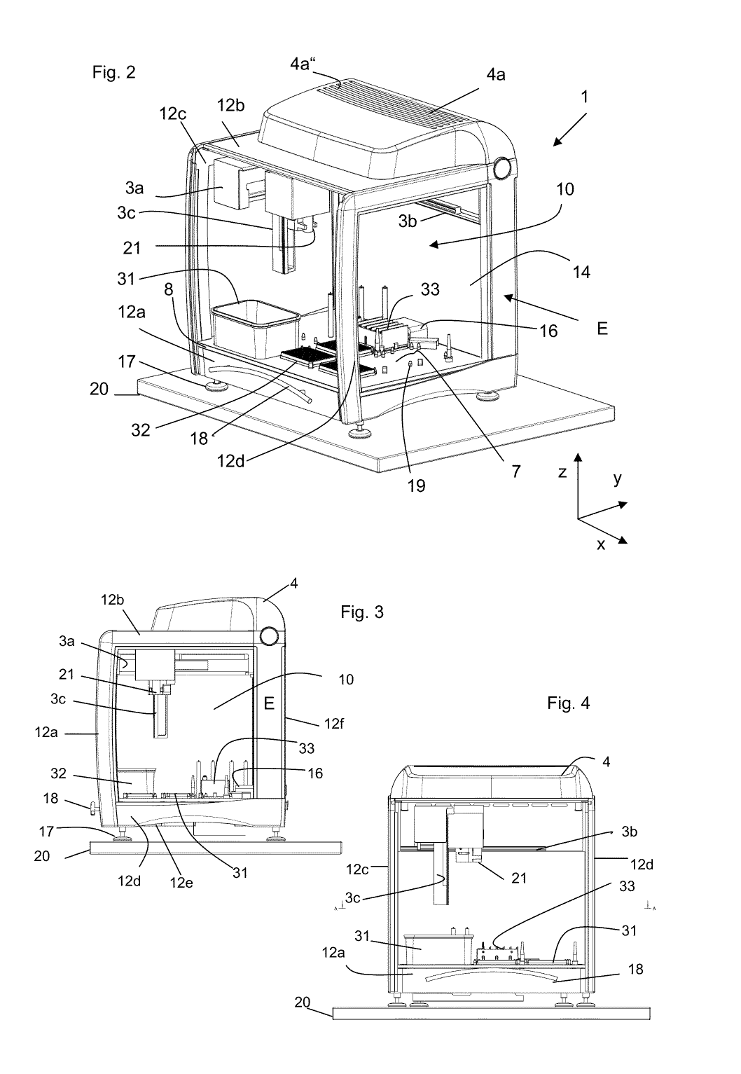

FIG. 2 shows the perspective view of another preferred embodiment of the apparatus according to the invention.

FIG. 3 shows a side view of the right side of the apparatus of FIG. 2.

FIG. 4 shows a front view of the apparatus of FIG. 2.

FIG. 5 shows a top view of the apparatus of FIG. 2.

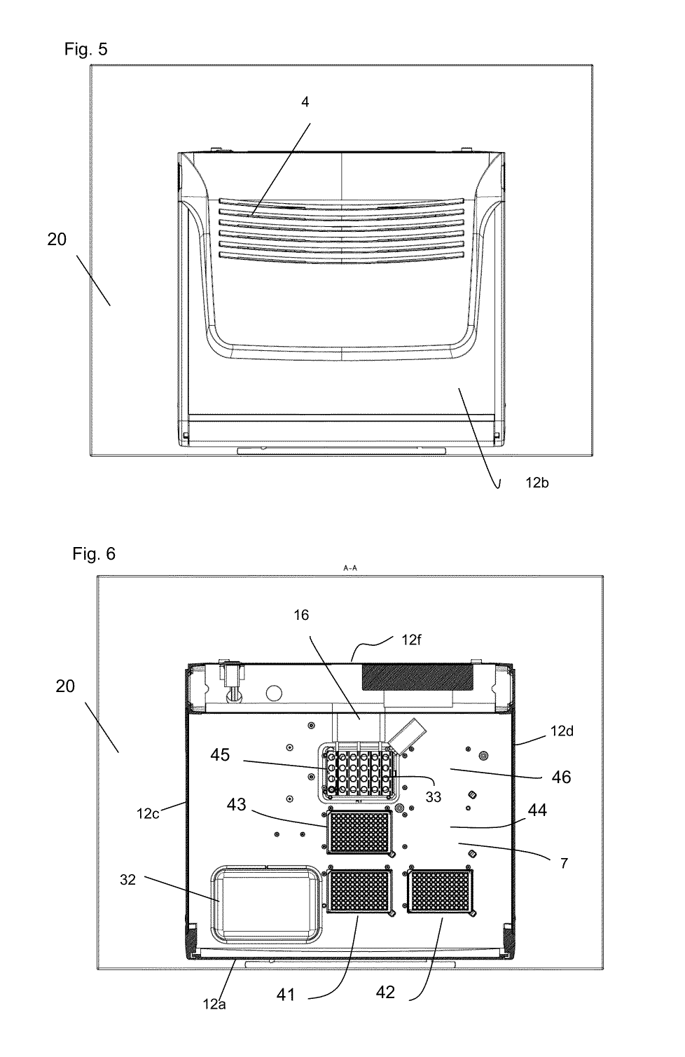

FIG. 6 shows another top view of the apparatus of FIG. 2, wherein the cover forming the top side of the apparatus is removed for showing the processing area of the processing space.

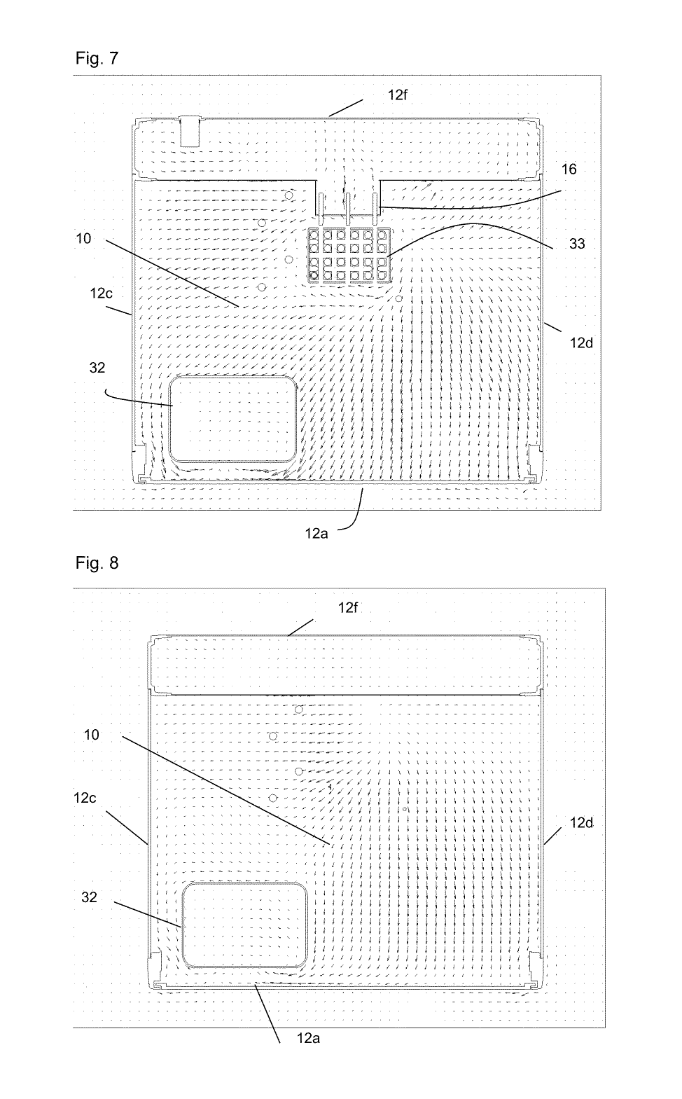

FIG. 7 shows a cross section in x-y-direction of the apparatus of FIG. 2 in a height of 20 mm above the processing area, and shows the flow field of the air, which forms, according to a mathematical simulation method, in the plane of the drawing during the activation of the air cleaning device, which is a decontamination device of the apparatus.

FIG. 8 shows a cross section in x-y-direction of the apparatus of FIG. 2 in a height of 20 mm above the sample holder element arranged in the processing area, and shows the flow field of the air, which forms, according to a mathematical simulation method, in the plane of the drawing during the activation of the air cleaning device, which is a decontamination device of the apparatus.

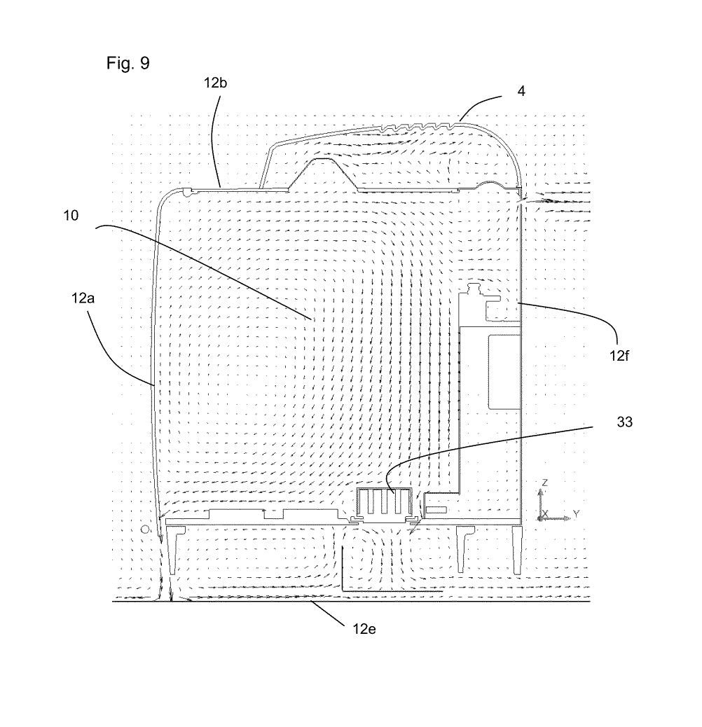

FIG. 9 shows a cross section in z-y-direction of the apparatus of FIG. 2 through the center of the processing area, and shows the flow field of the air, which forms, according to a mathematical simulation method, in the plane of the drawing during the activation of the air cleaning device, which is a decontamination device of the apparatus.

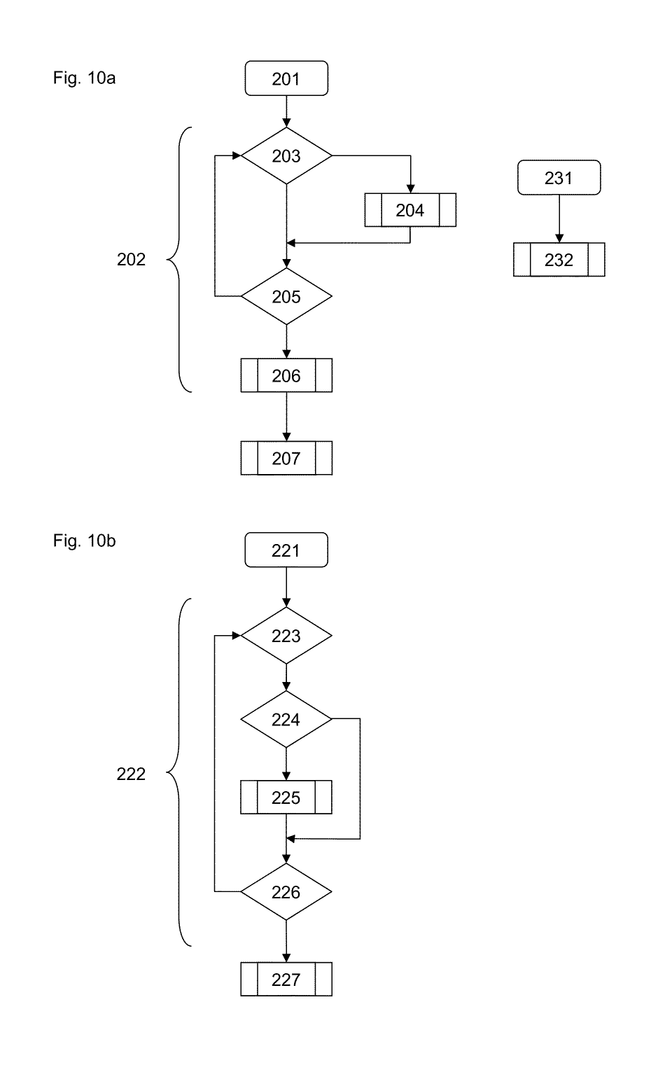

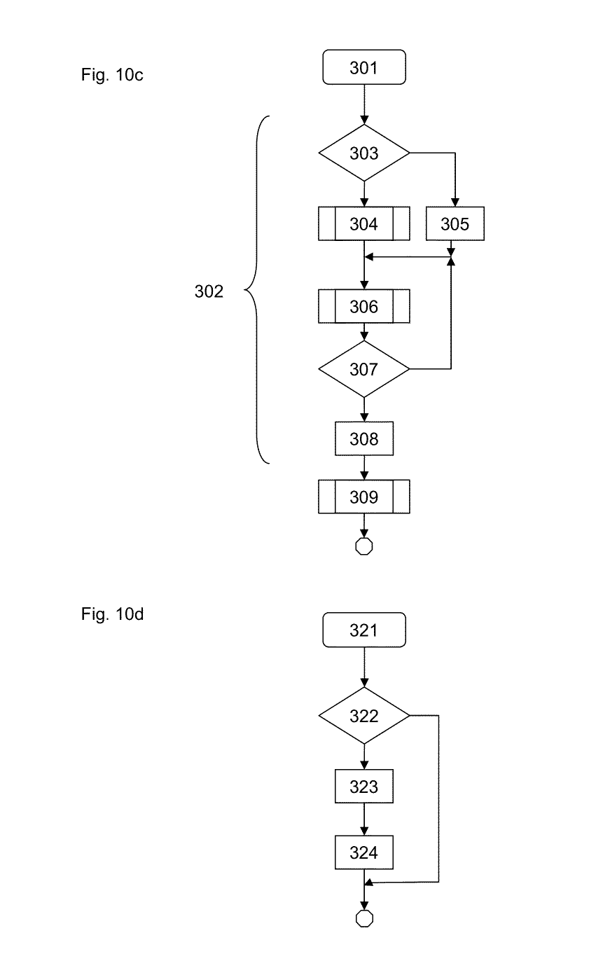

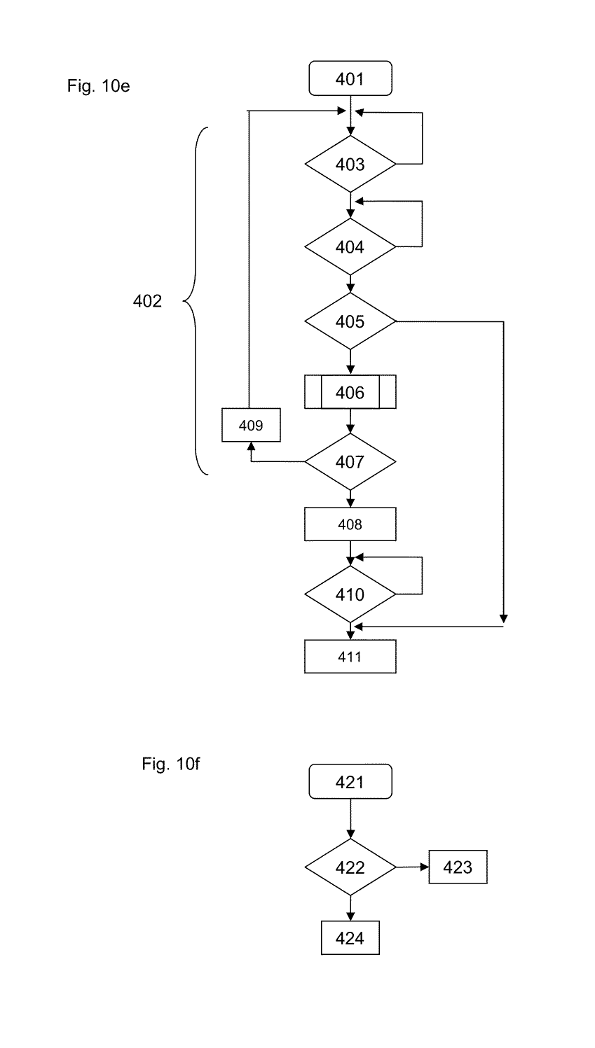

FIG. 10a shows a preferred embodiment of the method according to the invention, which uses a UV decontamination program.

FIG. 10b shows another preferred embodiment of the method according to the invention, which uses a UV decontamination program.

FIG. 10c shows a preferred embodiment of the method according to the invention, which uses a ventilation decontamination program.

FIG. 10d is related to the method of FIG. 10c and shows program steps for asking user defined parameters.

FIG. 10e shows a preferred embodiment of the method according to the invention, which uses a UV decontamination program.

FIG. 10f is related to the method of FIG. 10e and shows program steps for asking user defined parameters.

FIG. 1 shows the laboratory apparatus 1′ for the automated processing of liquid samples, in particular for the program controlled handling of liquid samples, having a socket section 13′, a housing 12′, an electronic control device 2′, which is adapted to process a program code for the program controlled processing of fluid samples. The apparatus 1′ has one processing space 10′ for receiving the fluid samples to be processed, an electronically controllable sample processing device 3′ for performing at least one program controlled process step on at least one sample, which can be arranged in the processing space, an electronically controllable decontamination device 4′ for cleaning at least a part of the processing space, wherein the control device 2′ has a control program 2a′, and a decontamination program 2c′, which is controlled by at least one method program 2b′, which is run by the control program. The decontamination device 4′ is configured to be controlled by the control device and the control device 2′ is configured to digitally control the decontamination device 4′. The digital control of the decontamination device 4′ allows for an efficient decontamination of the processing space 10′.

FIG. 2 shows the laboratory apparatus 1 for the automated processing of liquid samples, in particular for the program controlled handling of liquid samples. The apparatus 1 is a desktop device and is placed with its four sockets 17 on desktop 20. It has an electronic control device 2 (not shown), which is adapted to process a program code for the program controlled processing of fluid samples. The control device 2 is mounted in the control space, which is indicated by arrow E and is separated from the processing space 10 by a vertical wall 14. The control space also hosts the power electronics, which provide the appropriate voltage for the electrical components of the apparatus.

The apparatus 1 has one processing space 10 for receiving the fluid samples to be processed, an electronically controllable sample processing device 3 for performing at least one program controlled process step on at least one sample, which can be arranged in the processing space.

The apparatus 1 has a housing (12), which has a front side 12a, a back side 12f (not shown in FIG. 2) opposite to the front side, a top side 12b, a bottom side 12e (not shown in FIG. 2) opposite to the top side, and to opposing lateral sides 12c and 12d. The sides 12a, 12b and 12c are essentially formed by a material, which is transparent for visible light and intransparent for UV light, which material is preferably based on PMMA.

The front side 12a, which is formed essentially as a door element 12a, namely a sliding door 12a, which can be manually moved up and down, substantially along the z-axis of the Cartesian coordinate system. In the description of the present invention, the direction −z (minus z) refers to the direction of gravity, which is from up to down, and is a vertical direction. Any direction in parallel to the x-y-plane of the Cartesian coordinate system is referred to as horizontal direction. For the embodiment of the apparatus 1, the direction from the front to the back means the direction in y-direction of the Cartesian coordinate system, a direction from left to right means the direction in x-direction of the Cartesian coordinate system.

In FIG. 2, the closed position of the front door 12a is shown. In the closed position, a horizontally arranged gap 15 (not shown in FIG. 2) between the front door 12a and the bottom plate element 9 remains, which forms a ventilation channel 15, which connects the processing space with the environment. The gap substantially contributes, in the example of FIG. 2, to realize an air stream field in the processing space, where air is blown into the processing space in the back/top space, and the air is at least partly allowed to leave the processing space through gap 15. A similar gap 15b (not shown) is located between the waste container 31 and an opening in the bottom plate element 9. The gap 15b serves to remove aerosols and other contaminants from the processing space in a most directly way, which contaminants may form in the vicinity of the waste container 31, e.g. during the ejection of used pipette tips in the waste container.

The processing space 10 is confined by the front side 12a and the two lateral sides 12c and 12d as well as the wall 14, and the processing area 8, which is the upper side of the bottom plate element 9. The processing area 8 provides six processing stations 41, 42, 43, 44, 45, 46, 47 and 48. The processing stations are basically plane areas in the processing area 8. Pins 19 serve to align lab-ware at the processing station. The precise positioning allows for a precise robot-related addressing of the sample containers, e.g. wells of a microtiter plate 32, which are arranged in the present assembly, as an example, at processing stations 41, 42 and 43 (see top view in FIG. 6). A magnetic separation device 16 is arranged close to processing station 45, where a thermorack, i.e. a temperature controlled sample vessel holder is arranged. The magnetic fork (not shown) of the magnetic separation device 16 can enter/leave the thermorack 33 from the side, along the y-direction, to start/stop magnetic separation of magnetic particles in the sample solutions, which may be contained in the sample vessels in the thermorack.

The apparatus 1 has two different decontamination devices 4, an electronically controllable air cleaning device 4a, for cleaning the processing space, which is electronically and digitally controlled by the control device and which has a ventilation device 4a′. The ventilation device has three ventilators (not shown), which convey an air stream from outside of the apparatus into the processing space. At optimal performance of the ventilation device, the noise of the ventilation device is automatically driven at 3400 U/min of a ventilator, wherein the resulting noise is restricted to 55 dBA, in 1 m distance to the ventilation device 4. In FIG. 2, the ventilation slots 4a″ are visible, through which the environmental air enters the ventilation path, which connects the outside with the processing space 10.

The air cleaning device 4a also has an air filter device (not shown), here an HEPA filter, which filters the air in the ventilation path.

The apparatus has a further decontamination device 4, namely UV-lamp 4b, which is a tube (not shown). The UV light source is also electronically and digitally controlled by the control device. The UV light is mounted under the top side of the housing 12, for irradiating the processing area 7 and the lab-ware and components arranged in the processing area 7, as far as they are not masked by a UV-resistant cover element.

The control device 2 has a control program, and a decontamination program, which is controlled by at least one method program, which is run by the control program. The decontamination devices 4 are configured, respectively, to be controlled by the control device and the control device 2 is configured to digitally control the decontamination devices 4, respectively. The digital control of the decontamination devices 4 allows for an efficient decontamination of the processing space 10.

The apparatus 1 has a sample processing device 3, which has a Cartesian movement device, with three sliding elements 3a, 3b, 3c, which correspond to movements along the y, x, and z-axis of the Cartesian coordinate system, respectively. Electronically controllable linear motors are provided for precisely driving the movement along the required directions. This way, the mounting head 21 can be moved to any required accessible position in the processing space 10. The movement device is part of a robotic system of the sample processing device 3, which transports the mounting head 21, with any tool device, e.g. a pipetting head or a gripping head, connected to the mounting head 21, to the required position, by program control.

FIG. 10a shows a preferred embodiment of the method according to the invention, which uses a UV decontamination program, for irradiating the processing area locally, using a global UV source, e.g. a UV tube, and masking the areas which should not be irradiated. The decontamination program 202 is called by the method program during the method program (201) is executed. The method program can be interrupted to stop automatically processing liquid samples and to run the decontamination program. The method program calls the decontamination program 202 as a sub-program. After finishing the decontamination program 202, the method program 201 will continue to run, in step 207. The decontamination program 202 provides a subprogram UVPos(X) (step 203) to mask an area of processing station number X, by placing a UV-intransparent cover element over the area of a processing station X. In step 204, processing station X is covered; this function UVPos(X) is repeated in step 205 for all processing stations X=1 . . . n, n=4 . . . 15, which require being covered against UV irradiation. In step 206, the UV irradiation of the processing area is performed, except for the masked areas. This way, a local illumination is automatically achieved, without any user input required. However, it is possible that the user, initially, defines positions X to be masked, by operating a graphical user interface, i.e. a UV-related wizard asks the user to specify the positions X (step 231). Program parameters related to the positions X are defined in step 232.

FIG. 10b shows another preferred embodiment of the method according to the invention, which uses a UV decontamination program for irradiating the processing area locally, using a local UV source mounted at a UV-tool device, e.g. a UV-spot source, e.g. focused UV light or a UV beam, e.g. from a UV-LED, which irradiates the required positions x. The method 221 starts a decontamination program 222 for the local decontamination of the processing area, or of lab-ware arranged in the processing area. The function UV2Pos(x) is called in step 223, which moves the UV-tool device to position x and irradiates the position x for a predefined time period and with a predefined intensity (ste 224). This is repeated for all predefined positions x=1 . . . n (step 225). Then, the decontamination program ends and the next command of the method program is run. Also this way, a local illumination is automatically achieved, without any user input required.

FIG. 10c shows a preferred embodiment of the method according to the invention, which uses a ventilation decontamination program, which is run during a method program. The program controls the activity of an air cleaning device, which has at least one ventilation device in combination with a HEPA filter for filtering the air, which is transported into the processing space of the apparatus by the ventilation device. The method program can be interrupted to automatically stop processing liquid samples and to run the decontamination program (step 301). The method program calls the decontamination program 302 as a sub-program. After finishing the decontamination program 302, the method program 301 will continue to run, in step 309.

FIG. 10d is related to the method of FIG. 10c and shows program steps for asking user defined parameters during a sub-program of “ventilator determination”. The air cleaning device has a ventilation device, which has multiple ventilators. Each ventilator is arranged to produce an individual air stream, which runs through an individual section of the processing space. This arrangement results in a combined air stream inside the processing space. The ventilators are adapted to be controlled individually by the control device. The individual control of the multiple ventilators is performed automatically and is program controlled, in particular by running the method program in FIG. 10d. The user is allowed during running the method program in FIG. 10d, to choose at least one user parameter, which controls the activity status, i.e. the on/off status, of one ventilation device out of the multiple ventilation devices. The setup of the activity can be related to specific steps during running a method, e.g. by providing a predetermined activity parameter to each step of the method. It is also preferred that the user is allowed, in particular during running the method program, to choose at least one user parameter, which controls the intensity of the a ventilation device, in particular, the speed of the ventilator, e.g. measured in rounds per minute, in case that the ventilator is set active. The method program in FIG. 10d could be run during the programming of a method program, or can be run during the running of a method according to a method program.

It is preferred that the ventilation device has three individual ventilators, which are named “1”, “2” and “3”. Preferably, the ventilators are arranged in a top area of the housing of the apparatus, in particular in the top wall or the back wall. Preferably, the ventilators are arranged along a straight line, such that two of said ventilators are arranged vicinal, respectively, ventilator number 1 is arranged to ventilate a left section of the processing space, ventilator number 2 is arranged to ventilate a centre section of the processing space and ventilator number 3 is arranged to ventilate a right section of the processing space, wherein the directions “left” and “right” are determined with respect to a user standing in front of the front side of the apparatus.

The activity status of a ventilator can be coded by numbers A_{V} ranging from 0 to 6, wherein each number refers to a specific combination of active ventilators, while the residual ventilators are switched off: A_{V}=0 can mean that ventilator number 1 is active (i.e. “on”, at least during a specific step of the method or during the overall method), which means the an outer left section of the processing space is ventilated. Of course, this may also result in a weak air stream in the centre section and the right section of the processing space. A_{V}=1 can mean that ventilators number 1 and 2 are active, A_{V}=2 can mean that ventilators number 1, 2 and 3 are all active, A_{V}=3 can mean that ventilators number 2 and 3 are active, A_{V}=4 can mean that ventilators number 1 and 3 are active, A_{V}=5 can mean that ventilator number 3 is active, A_{V}=6 can mean that ventilator number 2 is active.

The intensity parameter can be user defined, which means that the intensity of an active ventilator can be set by the user. The intensity may be arbitrary defined within a range of intensities, or the intensity may chosen by the user from predetermined values, e.g. from two different levels of intensity, “weak” and “strong”.

In FIG. 10d, the user can determine whether a ventilator is set active and which intensity is assigned to the active ventilator during the performance of a respective method step. In step 321, the user is asked to set the ventilator settings. In step 322, the user is asked with reference to a specific section of the processing space, if a ventilation is desired or not. If yes, the program parameters are set, which set active the respective ventilators. Furthermore, the intensity of the respective ventilator is set up in step 324. In step 324, the sub-program “ventilator determination” is ended and the program returns to the point where setting up the method program is continued.

During running of a method program, e.g. the method program in FIG. 10c, or possibly during programming of the method program, the user is asked at a certain step 301 of the method whether any ventilator should be programmed to be active during the method. In step 303, a wizard, or the sub-program “ventilator determination” in FIG. 10d is started. The sub program returns to step 305, in case that any ventilator was set to be inactive during a step 306 of the method program. The sub program returns to step 304, in case that any ventilator was set to be active during a step 306 of the method program. The ventilators are set active, according to the ventilation settings performed according to FIG. 10d, and the method step 306 is performed according to the ventilation settings. Said method step can be, for example, a step of sample transfer by pipetting, manipulating samples and/or vessels by a tool, providing the tool with new pipette tips, dropping pipette tips, setting temperature by means of a temperature control device of the apparatus, and so on. In step 307, it is examined whether the ventilation should continue during the next work step 306, and if yes, the ventilation is continued. If no, the ventilation stops (step 308). In step 309, the running of the method, or the programming of the method, is continued.

FIG. 10f is related to the method of FIG. 10e and shows program steps for asking user defined parameters. A wizard program requires the user to input decontamination parameters. During a step 421 of the programming of any method, the user is asked at a specific step 422 to choose between an automatic irradiation of the processing area with UV light after finishing the method in step 424, for the purpose of decontamination of the processing space, or no automatic irradiation in step 423, wherein it is possible that a decontamination using UV light can—in addition or exclusively—be manually initiated by the user after the method has finished. The steps in FIG. 10f correspond to step 405 in FIG. 10e.

FIG. 10e shows a preferred embodiment of the method according to the invention, which uses a UV decontamination program, which is run after a method program was finished, in step 401.

In step 401, the method program, e.g. a method for the separation of nucleic acid, is finished. The apparatus waits, until the opening of the front wall door of the apparatus is detected (step 403). The apparatus continues to wait in case that no opening of the front wall door is detected. In case that an opening of the front wall door was detected, the apparatus also detects, if the front wall door was again closed, in step 404. Only in case that said closing is confirmed, the next step 405 is performed. Otherwise, the apparatus waits until the front wall door is closed again. In case that no automatic UV irradiation was chosen by using the wizard according to the steps in FIG. 10f, the UV light sources stay switched off. In case that an automatic UV irradiation was chosen, the processing surfaces is scanned by a sensor of the apparatus, in particular a height sensor, e.g. a confocal sensor, which detects, whether the processing area was cleaned up by the user and all articles have been removed, including tools, receptacles, consumables, in step 406. The confirmation of the processing space being emptied is a precondition for running the automatic UV irradiation, in step 408. In case that the processing area was detected to be not free, i.e. being partially occupied by article(s), the user is informed (step 409) either optically, e.g. by a signal on the display of the apparatus, and/or acoustically, and/or by Email or by SMS, that the processing area is not cleaned up. In case that the processing area is detected to be free, in step 407, the UV irradiation starts and continues, as long as no opening of the front door is detected. In case that an opening of the front wall door during irradiation is detected, in step 410, the apparatus provides the information that the predetermined time of UV irradiation is not finished. In case that the irradiation is not interrupted by an opening of the front wall door, the irradiation continues for the predetermined time period, and finally the UV light sources are switched off (step 411).

Using said methods according to the embodiment, the decontamination of the apparatus is optimized such that the sample processing using the apparatus is comfortable and becomes safe and reliable.

1. Laboratory apparatus for the automated processing of liquid samples, in particular for the program controlled handling of liquid samples, having
an electronic control device, which is adapted to process a program code for the program controlled processing of fluid samples,
at least one processing space for receiving the fluid samples to be processed,
at least one electronically controllable sample processing device for performing at least one program controlled process step on at least one sample, which is arranged in the processing space,
at least one electronically controllable decontamination device for cleaning at least a part of the processing space,
characterized in that
the decontamination device is configured to be controlled by the control device and
the control device is configured to digitally control the decontamination device.

2. Laboratory apparatus according to claim 1, wherein the decontamination device has an air cleaning device, which has at least one ventilation device and at least one filter device.

3. Laboratory apparatus according to claim 2, which has at least one ventilation pathway, wherein the processing space has a top space and a bottom space, as well as a front space and a back space, wherein the air cleaning device is arranged to transport air from the outside of the processing space via at least one ventilation pathway into the processing space, wherein the air cleaning device is arranged to connect the at least one ventilation pathway to the intersection volume, which is formed by the intersection of the top space and the back space.

4. Laboratory apparatus according to claim 1, wherein the decontamination device has an UV-light device, which is capable to emit UV light.

5. Laboratory apparatus according to claim 1, wherein the control device is configured to at least temporarily control the decontamination device.

6. Laboratory apparatus according to claim 1, wherein the control device is configured to control the decontamination device by processing a decontamination program.

7. Laboratory apparatus according to claim 1, wherein the control device is configured to control the decontamination device by processing the program code, in particular by processing a method program for performing the automated treatment of at least one sample, which is configured for performing at least one program controlled process step on at least one sample, which is arranged in the processing space, during the automated treatment of the sample.

8. Laboratory apparatus according to claim 7, wherein the control device is configured to automatically control the starting and running of the decontamination device during a time period, which is either
before, or,
during, or
after
the performance of at least one program controlled process step on at least one sample during the automated treatment of the sample.

9. Laboratory apparatus according to claim 1, wherein the control device is configured to use a user defined control parameter for controlling the decontamination device.

10. Laboratory apparatus according to claim 1, wherein the apparatus has a sensor device for sensing at least one operational parameter of the apparatus and to control the decontamination device in dependence on the at least one operational parameter.

11. Method of operating a laboratory apparatus, in particular the apparatus according to the invention, for the automated processing of liquid samples, in particular for the program controlled handling of liquid samples,
the apparatus having an electronic control device, which is adapted to process a program code for the program controlled processing of fluid samples, a processing space for receiving the fluid samples to be processed, at least one electronically controllable sample processing device for performing at least one program controlled process step on at least one sample, which is arranged in the processing space, and at least one electrically controllable decontamination device for cleaning at least a part of the processing space,
comprising the step of letting the control device digitally control the at least one decontamination device.
